(19) United States
(12) Patent Application Publication
(10) Pub. No.: US 2016/0116512 A1
(43) Pub. Date: Apr. 28, 2016

(54) METHOD AND SYSTEM FOR MONITORING ENERGY CONSUMPTION

(71) Applicant: KOREA ELECTRONICS TECHNOLOGY INSTITUTE, Seongnam-si (KR)

(72) Inventors: Young Min JI, Seoul (KR); Jeong Hoon KANG, Seoul (KR); Ho Jung LIM, Seoul (KR); Yoon Bong NAM, Seoul (KR)

(21) Appl. No.: 14/525,544

(22) Filed: Oct. 28, 2014

(51) Int. Cl. G01R 21/00; G01N 33/00; F24F 11/00

(52) U.S. Cl. CPC G01R 21/00; F24F 11/0034; G01N 33/004; F24F 2011/0035; F24F 2011/0047; F24F 2011/0094

(57) ABSTRACT

A method and system for monitoring energy consumption is provided. The method for monitoring energy consumption estimates a number of people in a room based on an indoor CO₂ concentration, and determines whether an amount of energy consumed indoors is proper or not based on the estimated number of people in the room. Accordingly, a waste of energy can be prevented by monitoring when a proper amount of energy is consumed in comparison with the number of people in a room. In addition, the number of people in the room can be easily estimated at low cost prior to determining whether energy consumption is proper or not.

### TECHNICAL FIELD OF THE INVENTION

The present invention relates generally to monitoring energy consumption, and more particularly, to a method and system for monitoring whether a proper amount of energy is consumed indoors.

### BACKGROUND OF THE INVENTION

The peak of power consumption in a national power crisis is closely related to weather. Since the use of an air conditioner/heater increases rapidly when it is very hot or cold, the record of the national peak is updated.

When the national power crisis is followed by a large-scale blackout, the blackout may cause inconvenience and chaos, which are nearly disasters, as well as serious economic damage. Therefore, in order to prevent these problems, there is a need for a method for monitoring whether electric power is properly consumed indoors, that is, whether cooling/heating is properly performed indoors.

To determine whether a proper amount of electric power is consumed indoors, the number of people in a room should be grasped first. However, most of the existing buildings cannot grasp the number of people in a room.

In order to grasp the number of people in a room, video equipment, many proximity sensors or many infrared ray (IR) sensors are required. However, there is a problem that either of them costs a lot of money.

Therefore, there is a demand for a method for easily grasping the number of people in a room at low cost and monitoring an improper situation where much electric energy is consumed for cooling or heating when there is no people in the room.

### SUMMARY OF THE INVENTION

To address the above-discussed deficiencies of the prior art, it is a primary aspect of the present invention to provide a method and system for easily grasping the number of people in a room at low cost and monitoring whether a proper amount of energy is consumed in comparison with the number of people in the room in order to prevent a waste of energy.

According to one aspect of the present invention, a method for monitoring energy consumption includes: measuring an indoor CO₂ concentration; estimating a number of people in a room based on the CO₂ concentration; grasping an amount of energy consumed indoors; and determining whether the amount of energy consumed indoors is proper or not based on the estimated number of people in the room.

The estimating may include estimating the number of people in the room based on an increase rate of the CO₂ concentration.

The estimating may include, when the increase rate of the CO₂ concentration increases after opening/closing of an entrance door is detected, estimating that the number of people in the room increases, and, when the increase rate of the CO₂ concentration decreases after opening/closing of the entrance door is detected, estimating that the number of people in the room decreases.

The method may further include measuring an indoor O₂ concentration, and the estimating may include, when (an increase rate of the CO₂ concentration)/(a reduction rate of the O₂ concentration) falls within a predetermined range, regarding a change in the increase rate of the CO₂ concentration as being caused by a change in the number of people in the room.

The method may further include, when the amount of energy consumed indoors exceeds a threshold value in comparison with the number of people in the room, outputting a warning message.

The amount of energy consumed indoors may be an amount of electric power consumed indoors.

According to another aspect of the present invention, a system for monitoring energy consumption includes: a sensor configured to measure an indoor CO₂ concentration; a measuring unit configured to measure an amount of energy consumed indoors; and a server configured to estimate a number of people in a room based on the indoor CO₂ concentration measured by the sensor, and determine whether the amount of energy consumed indoors, which is measured by the measuring unit, is proper or not based on the estimated number of people in the room.

According to exemplary embodiments of the present invention as described above, a waste of energy can be prevented by monitoring when a proper amount of energy is consumed in comparison with the number of people in a room. In addition, the number of people in the room can be easily estimated at low cost prior to determining whether energy consumption is proper or not.

### BRIEF DESCRIPTION OF THE DRAWINGS

For a more complete understanding of the present disclosure and its advantages, reference is now made to the following description taken in conjunction with the accompanying drawings, in which like reference numerals represent like parts:

FIG. 1 is a block diagram showing an energy consumption monitoring system according to an exemplary embodiment of the present invention;

FIG. 2 is a flowchart to explain an energy consumption monitoring method according to another exemplary embodiment of the present invention;

FIGS. 3 and 4 are views to explain a process of estimating the number of people in a room as explained in FIG. 2 in more detail; and

FIGS. 5 and 6 are views to explain a process of determining whether electric power is properly consumed indoors as explained in FIG. 2 in more detail.

### DETAILED DESCRIPTION OF THE INVENTION

Reference will now be made in detail to the embodiment of the present general inventive concept, examples of which are illustrated in the accompanying drawings, wherein like reference numerals refer to the like elements throughout. The embodiment is described below in order to explain the present general inventive concept by referring to the drawings.

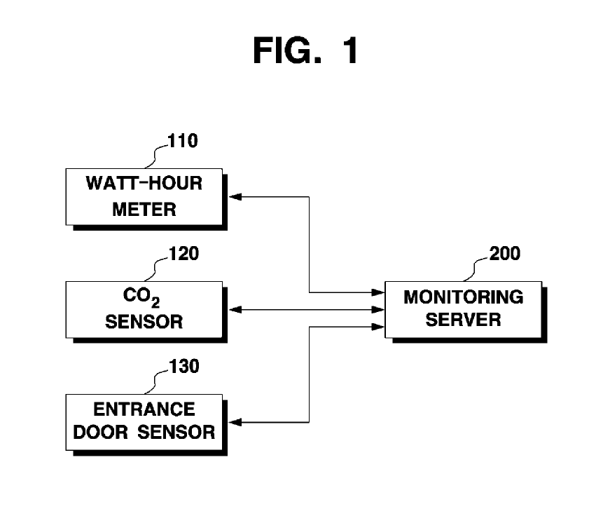

FIG. 1 is a block diagram showing an energy consumption monitoring system according to an exemplary embodiment of the present invention. The energy consumption monitoring system according to an exemplary embodiment includes a watt-hour meter 110, a CO₂ sensor 120, an entrance door sensor 130, and a monitoring server 200, as shown in FIG. 1.

The watt-hour meter 110, the CO₂ sensor 120, and the entrance door sensor 130 are all communicably connected to the monitoring server 200. Any connecting method such as wire connection or wireless connection may be used and there is no limit to the communication method.

The watt-hour meter 110 is a device for measuring an amount of electric power consumed indoors. The watt-hour meter 110 may be a device that is directly installed by electric power corporation or may be a product that is certified by the corporation.

The CO₂ sensor 120 is a sensor which measures indoor CO₂ concentration and transmits a result of the measuring to the monitoring server 200. The CO₂ sensor 120 may be implemented by using a single sensor or a plurality of sensors.

When the CO₂ sensor 120 is implemented by using a plurality of sensors, the sensors may be installed at proper indoor locations and the indoor CO₂ concentration may be obtained based on an average of measured results. Alternatively, different weights (Σ weights=1) may be assigned to the measured results of the different sensors. For example, a weight to be assigned to a result measured by a sensor located at the center may be greater than a weight to be assigned to a result measured by a sensor located at an edge.

The entrance door sensor 130 is a sensor for detecting opening/closing of an entrance door through which people come in or out, and the detected state of the entrance door (opening or closing) may be transmitted to the monitoring server 200.

The monitoring server 200 estimates the number of people in a room based on the results of measuring/detecting collected by the watt-hour meter 110, the CO₂ sensor 120, and the entrance door sensor 130, and monitors whether electric power is properly consumed indoors or not based on the number of people, and notifies a manager of the result.

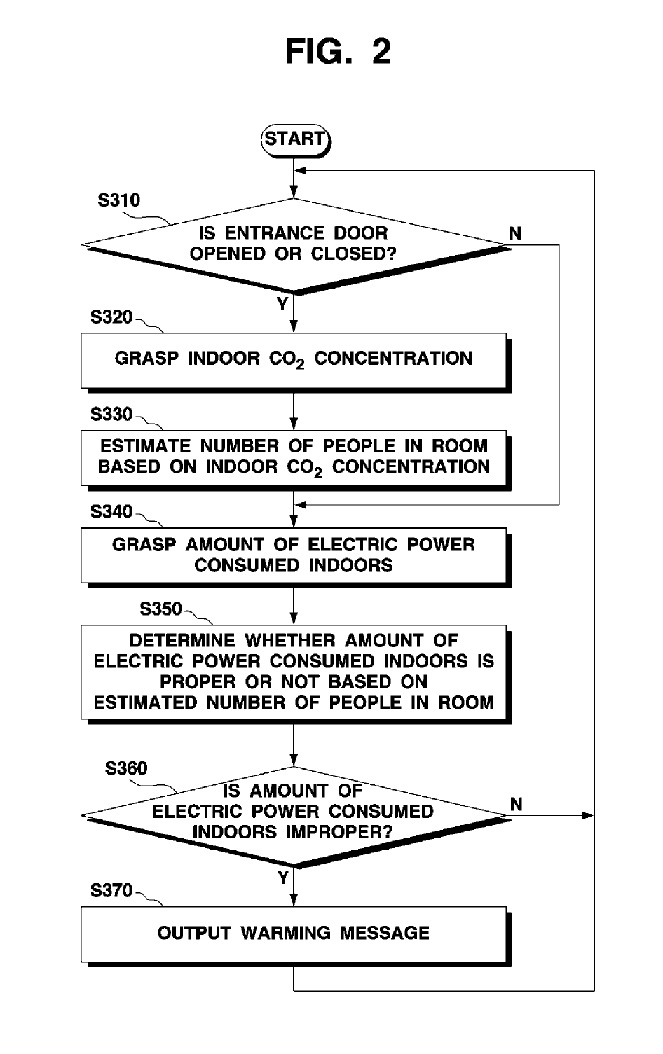

Hereinafter, a process of monitoring indoor electric power consumption by means of the monitoring server 200 will be explained with reference to FIG. 2. FIG. 2 is a flowchart to explain a method for monitoring energy consumption according to another exemplary embodiment of the present invention.

As shown in FIG. 2, when opening/closing of the entrance door is detected by the entrance door sensor 130 (S310-Y), the monitoring server 200 measures indoor CO₂ concentration for a predetermined time (S320), and estimates the number of people in the room based on the measured indoor CO₂ concentration (S330).

In step S320, the indoor CO₂ concentration may be measured by the monitoring server 200 waking up the CO₂ sensor 120 in a sleep mode and instructing to measure the CO₂ concentration, and receiving the measured CO₂ concentration from the CO₂ sensor 120.

In this embodiment, in step S330, the number of people in the room may be estimated only when the opening/closing of the entrance door is detected by the entrance door sensor 130. A change in the number of people in the room requires that the entrance door should be opened or closed. Therefore, when the entrance door is neither opened nor closed, the number of people in the room is regarded as being maintained as it is. Accordingly, the CO₂ sensor 120 measures the CO₂ concentration only when the number of people in the room is expected to change and thus electric power consumed by the CO₂ sensor 120 can be minimized.

In step S330, the number of people in the room is estimated based on an increase rate of the CO₂ concentration. That is, when the increase rate of the CO₂ concentration increases, it is estimated that the number of people in the room increases, and, when the increase rate of the CO₂ concentration decreases, it is estimated that the number of people in the room decreases.

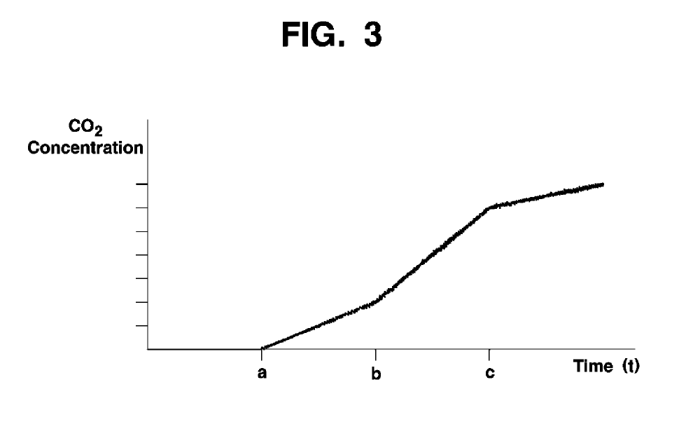

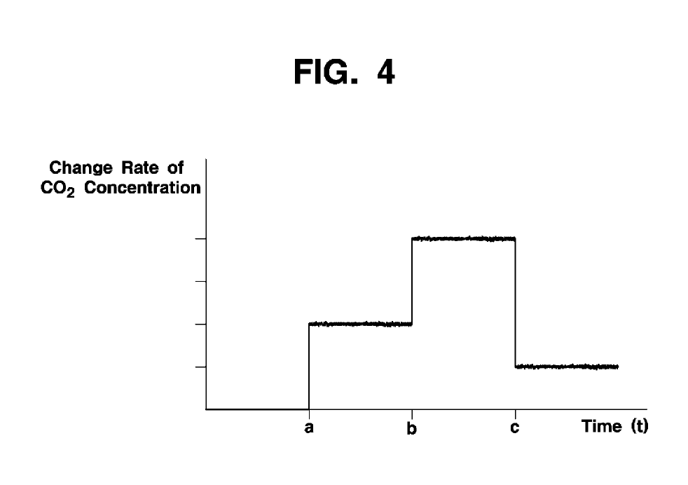

Referring to FIGS. 3 and 4, the process of estimating the number of people in the room will be explained in more detail. FIG. 3 is a graph showing a change in indoor CO₂.

In FIG. 3, points of time ‘a’, ‘b’, and ‘c’ are points of time at which the entrance door is opened or closed and the number of people in the room is changed. Specifically, at the point of time ‘a’, people enter the room and thus indoor CO₂ is generated. At the point of time ‘b’, the number of people in the room increases and thus the increase rate of the indoor CO₂ concentration increases. However, at the point of time ‘c’, the number of people in the room decreases and thus the increase rate of the indoor CO₂ concentration decreases.

FIG. 4 is a graph showing the increase rate of the CO₂ concentration of FIG. 3. It can be seen from the graph of FIG. 4 that the number of people in the room is changed with time.

Referring to FIGS. 3 and 4, when the increase rate of the indoor CO₂ concentration increases after the opening/closing of the entrance door is detected, it is estimated that the number of people in the room increases, and, when the increase rate of the indoor CO₂ concentration decreases after the opening/closing of the entrance door is detected, it is estimated that the number of people in the room decreases.

In FIG. 3, the graph shows the CO₂ concentration at all of the points of time. However, this is for the convenience of explanation. The monitoring server 200 is only required to grasp the increase rate of the indoor CO₂ concentration. Therefore, the indoor CO₂ concentration has only to be measured for a predetermined time after the opening/closing of the entrance door is detected. This is because the monitoring server 200 can grasp the increase rate of the indoor CO₂ concentration just by doing so.

Thereafter, the monitoring server 200 grasps an amount of electric power consumed in the room (S340). In step S340, the amount of electric power consumed in the room may be grasped by the monitoring server 200 requesting a current status of indoor electric power consumption from the watt-hour meter 110 and receiving the current status of the indoor electric power consumption.

Next, the monitoring server 200 determines whether the amount of electric power grasped in step S340 is proper or not based on the number of people measured in step S330 (S350).

Specifically, in step S350, the monitoring server 200 determines whether the amount of electric power consumed in the room exceeds a threshold value or not in comparison with the number of people in the room, and determines whether the amount of electric power consumed in the room is proper or not. As the number of people in the room decreases, the threshold value decrease, and, as the number of people in the room increases, the threshold value increases.

When it is determined that the amount of electric power consumed in the room is improper in step S350 (S360-Y), the monitoring server 200 outputs a warning message to notify the manager (S370). In step S370, the warning message may be transmitted to a terminal of the manager.

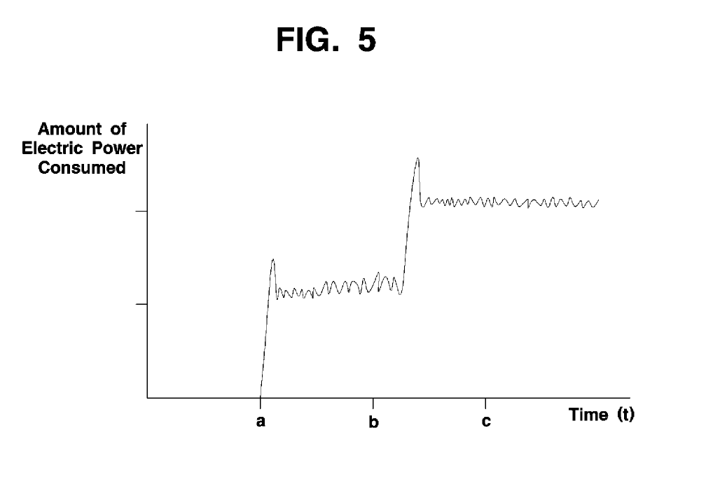

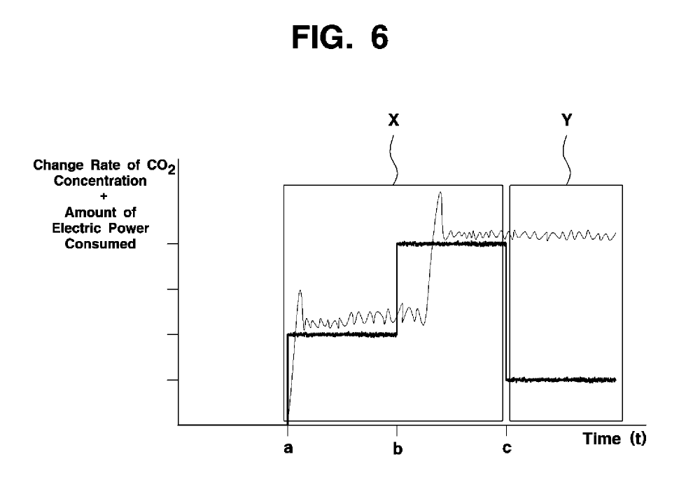

FIG. 5 illustrates a current status of electric power consumption. FIG. 6 is a graph showing FIGS. 4 and 5 altogether. As shown in FIG. 6, the amount of electric power consumed is proper at the points of time before the point of time ‘c’ (X), but the amount of electric power consumed is improper at the points of time after the point of time ‘c’ (Y). In this state, the warning message is output in step S370. This is because the amount of electric power is not reduced even when the number of people in the room decreases.

Up to now, the method and system for estimating the number of people in a room based on indoor CO₂ concentration and monitoring whether electric power is properly consumed indoors based on the number of people according to an exemplary embodiment has been described.

In the above-described exemplary embodiment, the amount of electric power consumed is monitored. However, the technical idea of the present invention can be applied when an amount of other energy consumed (gas, oil, water, etc.) is monitored.

In addition, indoor O₂ concentration may be measured in addition to the indoor CO₂ concentration. This is to identify whether the increase in the CO₂ concentration is caused by the use of a CO₂ generating device such as a gas range or increase of the number of people in the room.

Specifically, a value (range) of (an increase rate of CO₂ concentration)/(a reduction rate of O₂ concentration) caused by a human's breathing is calculated, and a value (range) of (an increase rate of CO₂ concentration)(a reduction rate of O₂ concentration) caused by the use of a gas range is calculated. These values are stored in the monitoring sever 200. It may be determined whether increase in the indoor CO₂ concentration is caused by increase in the number of people in the room or not by grasping which value (range) a change in the increase rate of the CO₂ concentration belongs to.

Although the present disclosure has been described with an exemplary embodiment, various changes and modifications may be suggested to one skilled in the art. It is intended that the present disclosure encompass such changes and modifications as fall within the scope of the appended claims.

What is claimed is:

1. A method for monitoring energy consumption, the method comprising:
measuring an indoor CO₂ concentration;
estimating a number of people in a room based on the CO₂ concentration;
grasping an amount of energy consumed indoors; and
determining whether the amount of energy consumed indoors is proper or not based on the estimated number of people in the room.

2. The method of claim 1, wherein the estimating comprises estimating the number of people in the room based on an increase rate of the CO₂ concentration.

3. The method of claim 2, wherein the estimating comprises, when the increase rate of the CO₂ concentration increases after opening/closing of an entrance door is detected, estimating that the number of people in the room increases, and, when the increase rate of the CO₂ concentration decreases after opening/closing of the entrance door is detected, estimating that the number of people in the room decreases.

4. The method of claim 2, further comprising measuring an indoor O₂ concentration, and
wherein the estimating comprises, when (an increase rate of the CO₂ concentration)/(a reduction rate of the O₂ concentration) falls within a predetermined range, regarding a change in the increase rate of the CO₂ concentration as being caused by a change in the number of people in the room.

5. The method of claim 1, further comprising, when the amount of energy consumed indoors exceeds a threshold value in comparison with the number of people in the room, outputting a warning message.

6. The method of claim 1, wherein the amount of energy consumed indoors is an amount of electric power consumed indoors.

7. A system for monitoring energy consumption comprising:
a sensor configured to measure an indoor CO₂ concentration;
a measuring unit configured to measure an amount of energy consumed indoors; and
a server configured to estimate a number of people in a room based on the indoor CO₂ concentration measured by the sensor, and determine whether the amount of energy consumed indoors, which is measured by the measuring unit, is proper or not based on the estimated number of people in the room.
